(19) United States
(12) Patent Application Publication
(10) Pub. No.: US 2004/0034023 A1
(43) Pub. Date: Feb. 19, 2004

(54) Use of polycyclic aromatic compounds for making medicines capable of inhibiting telomerase

(76) Inventors: Jean-Louis Mergny, Villejuif (FR); Laurent Lacroix, Paris (FR); Marie-Paule Teulade-Fichou, Paris (FR); Jean-Pierre Vigneron, Boissy (FR); Jean-Marie Lehn, Strasbourg (FR); Claude Helene, Paris (FR)

Correspondence Address: CONLEY ROSE, P.C., P. O. BOX 3267, HOUSTON, TX, 77253-3267, US

(21) Appl. No.: 10/343,616

(22) PCT Filed: Jul. 30, 2001
(86) PCT No.: PCT/FR01/02492

(30) Foreign Application Priority Data
Aug. 2, 2000 (FR) 00/10218

(51) Int. Cl. A01N 43/66; A61K 31/4745

(52) U.S. Cl. 514/241; 514/298; 514/313; 534/551; 534/800; 536/32; 546/108; 514/280

(57) ABSTRACT

The invention concerns the use of aromatic compounds capable of binding with G-quadruplex structures to produce medicines with anti-telomerase effect. Said compounds correspond to formula (I) wherein: R₁, R₂ and R₃, identical or different, represent a hydrogen atom, or a —CH₂—NH—(CH₂)ₙ—X group, wherein n is an integer from 2 to 4, and X is selected among —NH₂—, —N(CH₃)₂ radicals, a heterocyclic radical such as piperidyl, imidazolyl, morpholinyl radical, or an indole-type condensed heterocyclic radical, —Z represents CH or N, each compound comprising two nitrogen atoms in the Z positions. The invention is useful for producing anticancer medicines.

The subject of the invention is the use of polycyclic aromatic compounds to prepare medicaments having telomerase-inhibiting properties and able to be used, in particular, for the treatment of cancers.

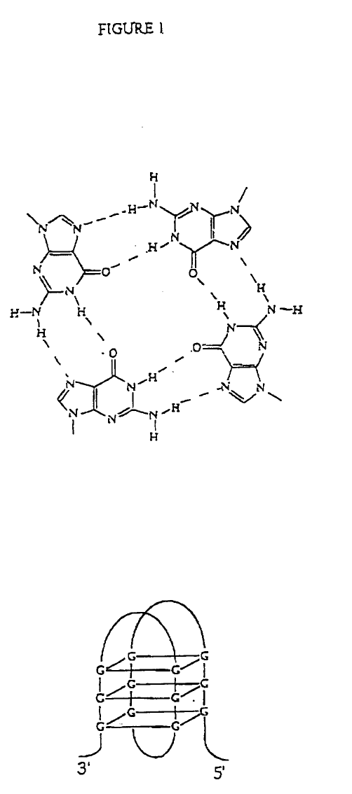

The DNA of the telomeres in humans is essentially constituted by a double strand and contains repeated TTAGGG/CCCTAA units. The ends, on the other hand, are single-stranded with a 3′ region rich in G units. This single-stranded DNA can adopt a 4-strand structure involving G-quartets as illustrated in FIG. 1, or can form a T-shaped loop.

From the biological point of view, telomerase permits the addition of these repeated DNA sequences to the end of the telomere, during cell division. Through this action, the telomerase makes the cell immortal. In the absence of this enzymatic activity, the cell actually loses from 100 to 150 bases with each division, which renders it rapidly senescent. Upon the appearance of rapidly dividing cancer cells, it has been shown that these cells contained telomeres kept at a stable length during the cell division. In these cancer cells, it appeared that the telomerase was strongly activated and that it permitted the addition of repeated units of telomeric sequences to the end of the telomere and thus permitted the preservation of the length of the telomere in the cancer cells. More than 85% of the cancer cells present positive tests for the presence of telomerase whereas the great majority of the somatic cells do not display this characteristic.

Telomerase is thus a greatly desired target for the treatment of cancer cells. The obvious first approach to the problem of blocking telomerase consisted of the use of nucleotide structures (Chen. et al., Proc. Natl. Acad. Sci. USA 1996, 93 (7), 2635-2639). Among the non-nucleotide compounds that have been used in the prior art, there may be cited the diaminoanthraquinones (Sun et al. J. Med. Chem. 40(14), 2113-6) or the diethyloxadicarbocyanines (Wheelhouse R. T. et al J. Am. Chem. Soc. 1998 (120) 3261-2). Application WO 99/40087 describes the use of compounds capable of interacting with the G-quadruplex structures mentioned above. These are perylene compounds and carbocyanines containing at least seven cycles, two of which are heterocycles.

The work of inventors has shown that, in a surprising manner, aromatic compounds with a planar structure in the shape of a crescent, i.e. dibenzophenanthrolines, some of which are known for their triplex-stabilizing effect, allowed a result to be obtained which was at least equivalent with structures that were much less complicated from the chemical point of view.

The expansion of this work also made it possible to develop novel aromatic compounds of this type that for their part are also capable of fixing themselves to the G quadruplex structure, thus displaying a telomerase-inhibiting activity.

According to one of its aspects, the invention thus relates to the use of compounds of dibenzophenanthrolines to prepare medicaments having an anti-telomerase effect.

Its aim is also, according to another aspect, to provide novel dibenzophenanthrolines and their use as an active ingredient of medicaments.

The use of aromatic compounds capable of binding to G-quadruplex structures in order to prepare medicaments having an anti-telomerase effect according to the invention is characterized in that the said compounds conform to Formula (I)

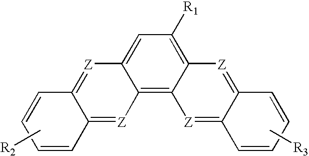

in which

R₁, R₂ and R₃, identical to or different from one another, represent a hydrogen atom, or a —CH₂—NH—(CH₂)ₙ—X group, in which n is an integer from 2 to 4, and X is chosen from among the —NH₂, —N(CH₃)₂ radicals, a heterocyclic radical such as the piperidyl, imidazolyl, morpholinyl radical, or a condensed heterocyclic radical of indole type.

Z represents CH or N, each compound containing two nitrogen atoms in the 4 “Z” positions.

The invention relates in particular to the use of the compound of Formula (II)

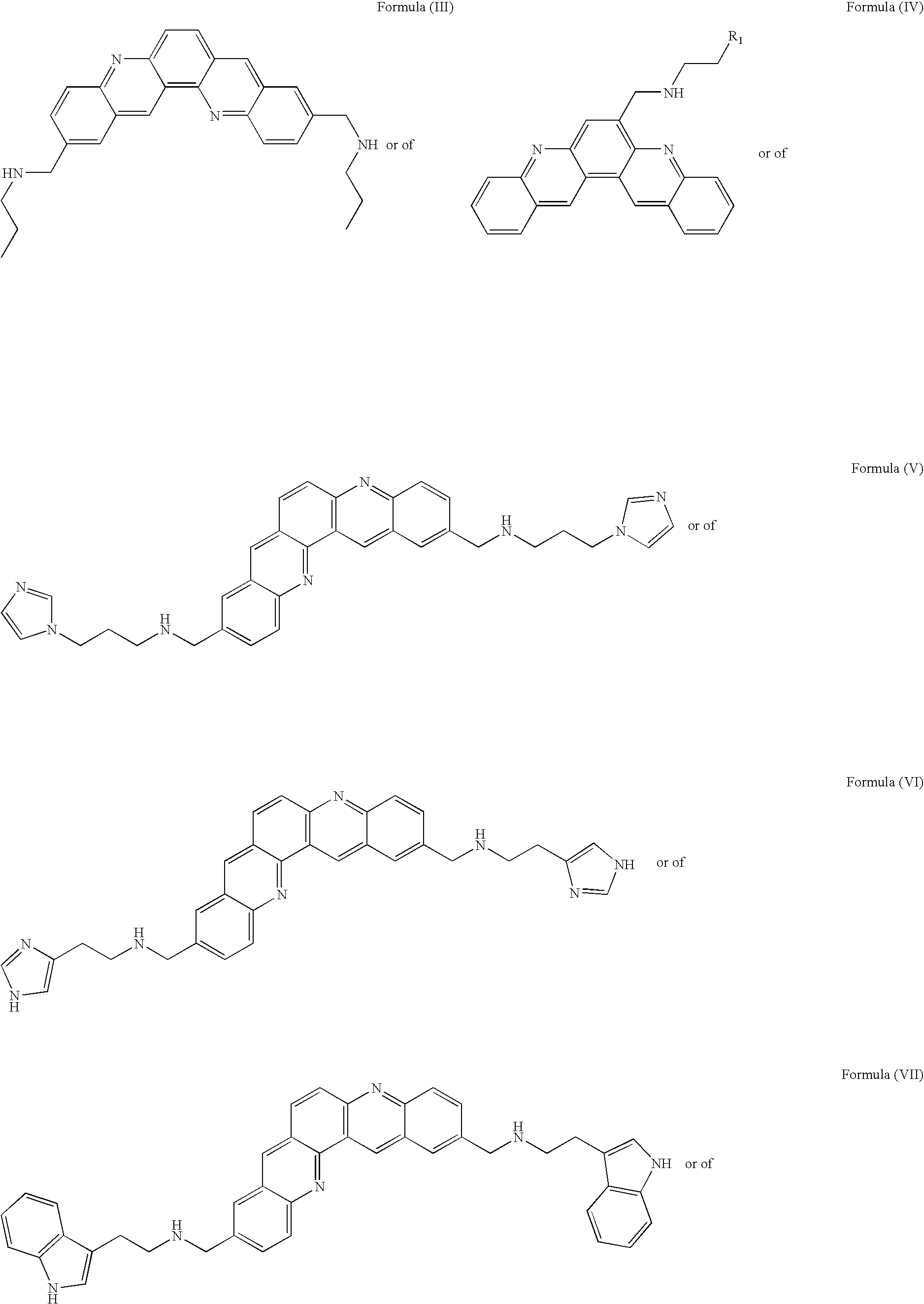

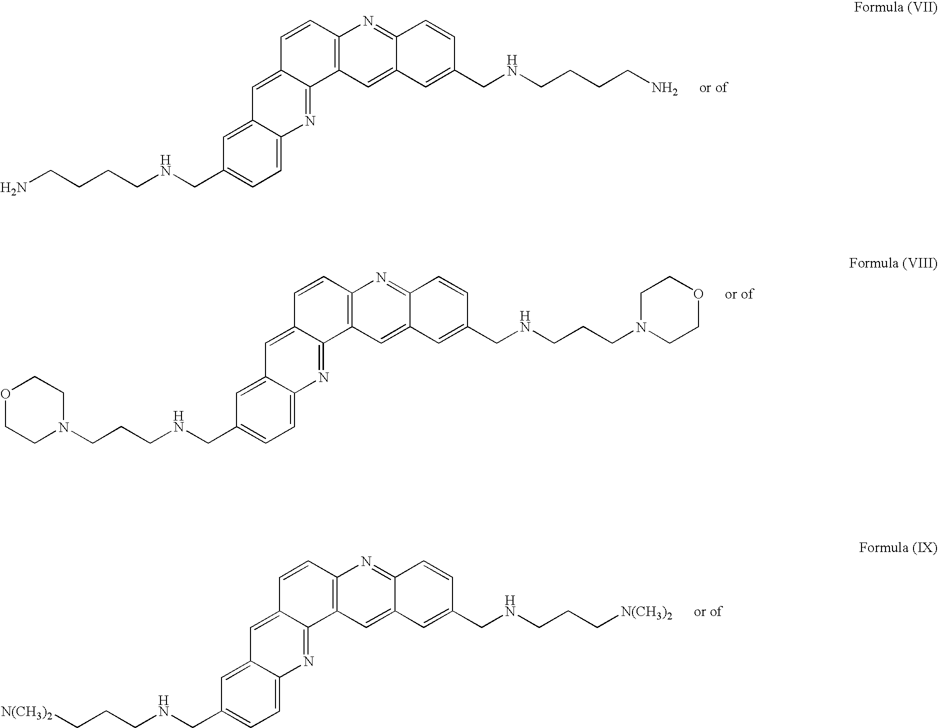

The compounds of Formula (I) used, according to the invention, as active ingredients of medicaments are characterized in that they are capable of increasing the melting point (Tm) of G-quadruplex by 2 to 20° C. at a concentration of 1 μM, in particular by 7 to 20° C. for those of Formulae (IV) to (IX). This increase in Tm is correlated to that of their anti-telomerase effect, in vitro.

It will be noted with interest that the IC₅₀ values of these compounds are advantageously below 3 μM and even below 1 μM for a number of them.

In the light of the therapeutic applications referred to, it is interesting to note that the compounds of Formula (I) also display dissociation constants of the order of 10⁻⁸ M and thus bind very strongly to the G-quadruplex structures whereas the dissociation constants of the quadruplex ligands known to date are of the order of 10⁻⁶ to 10⁻⁵ M.

According to another aspect, the invention also relates, as novel products, to the polycyclic aromatic compounds of Formula (X)

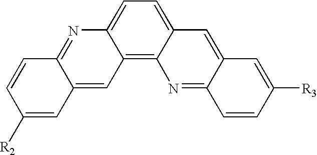

in which

R₁, R₂ and R₃, identical to or different from one another, represent a hydrogen atom, or a —CH₂—NH—(CH₂)ₙ—X group, in which n is an integer from 2 to 4, and X is chosen from among the —NH₂, —N(CH₃)₂ radicals, a heterocyclic radical such as the piperidyl, imidazolyl, morpholinyl radical, or a condensed heterocyclic radical of indole type.

The study of these products, according to the tests reported in the examples, has shown that they are capable of stabilizing the G-quadruplex structures of the telomeres and can consequently be used for the development of medicaments having an anti-telomerase effect, in particular anti-tumoral medicaments.

The invention thus also relates to pharmaceutical compositions containing a therapeutically effective quantity of at least one of these novel compounds in combination with a pharmaceutically inert vehicle.

The medicaments according to the invention or prepared according to the invention are of quite particular benefit for the treatment of cancers. They are developed in appropriate forms for the method of administration that is desired for this type of treatment. This most generally means forms for an administration by the oral, nasal, buccal, injectable, parenteral, rectal, vaginal or topical route.

For oral administration, compositions of medicaments are formulated in order to obtain, in standard manner, compressed tablets, sugar-coated tablets, pills, gels, capsules and the like. The unit dosage will be adapted by a person skilled in the art in order to achieve the desired therapeutic effect.

For administration by injection, sterile or sterilizable solutions are prepared that can be administered by the subcutaneous, intravenous, intradermal, intramuscular or parenteral route. These solutions contain the requisite quantity, for the desired effect, of active ingredient.

These administrations can be carried out once or more often.

The other forms of administration are advantageously prepared according to the customary galenic formulations.

Other characteristic features and advantages of the invention are given in the following examples in which reference will be made to FIGS. 1 to 3, which represent, respectively.

FIG. 1A, a G-quartet structure and FIG. 1B an intramolecular quadruplex,

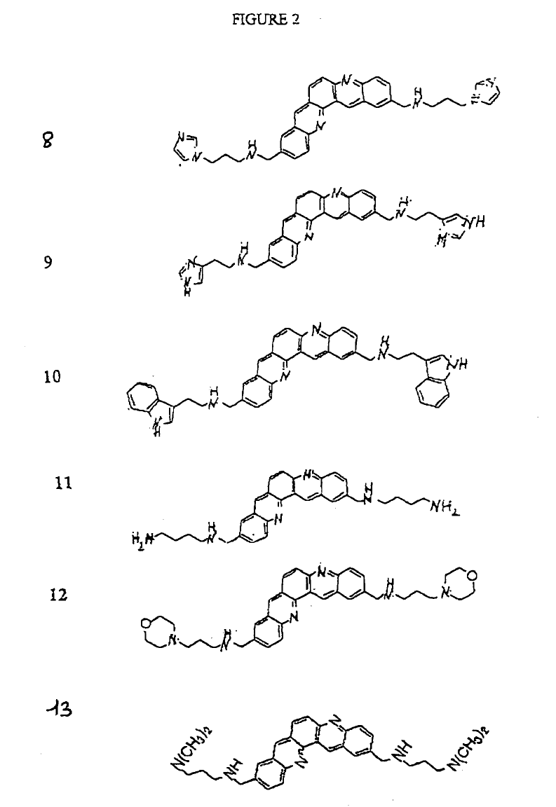

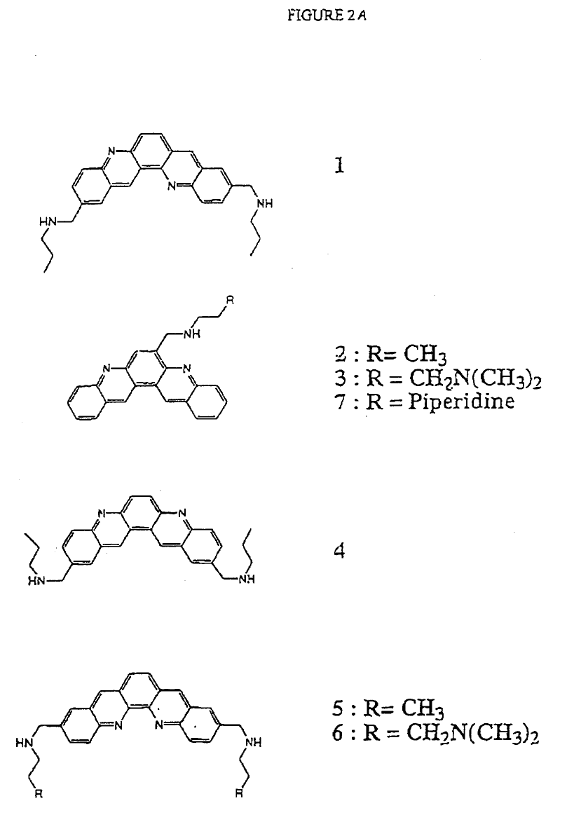

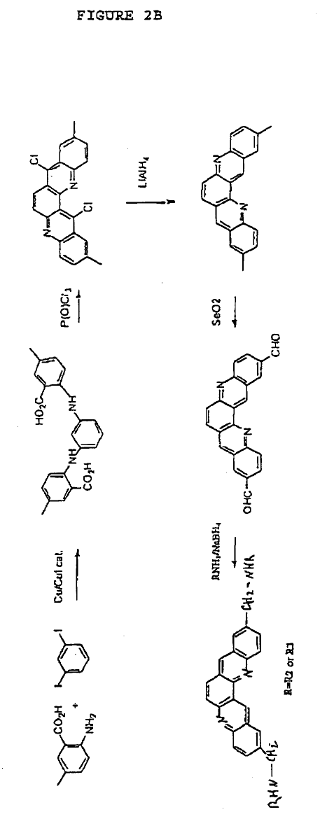

FIG. 2A, the chemical formulae of dibenzophenanthrolines tested as ligands of G-quartets and Formula 2B, the synthesis diagram for derivatives of dibenzophenanthrolines according to the invention, following the protocol given by Baudoin et al in J.O.C., 1997, 62, 5448,

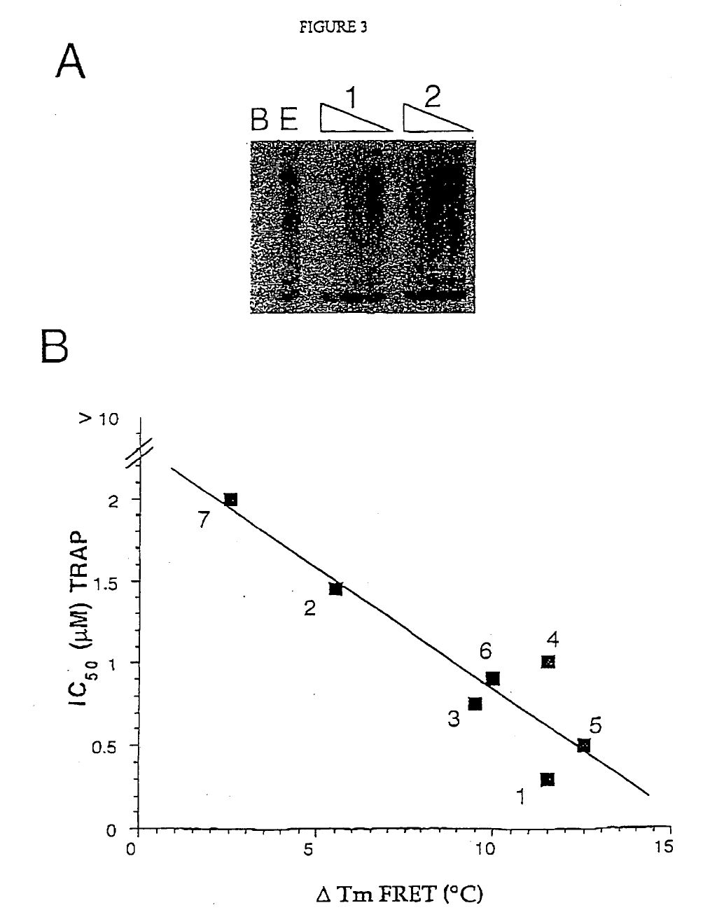

FIG. 3A, the inhibition of the telomerase by the diphenanthrolines according to the invention and

FIG. 3B, the correlation of this inhibition with the G-quartet stabilization.

Materials and Methods

Oligonucleotides

All the oligonucleotides, modified or not, were synthesized by Eurogentec S. A., Seraing, Belgium. For the fluorescence studies, a fluorescein molecule is attached to the 5′ end of the oligonucleotide used, and a tetramethylrhodamine molecule to its 3′ end. The concentration of the specimens is verified by spectrophotometry, recording the absorbance spectrum between 220 and 700 nm and using its molar extinction coefficient as reported by the supplier.

Dibenzophenanthrolines

The synthesis of compounds 1 to 6 was carried out as described by Baudoin et al in Chemistry: a European Journal 4, 1504-1508 (1998). Compound 7, namely 6-[2-piperidin-1-yl)ethyl)aminomethyl]dibenzo[b,j]4,7]phenanthroline, was synthesized in accordance with the protocol described for compounds 2 and 3 by Baudoin et al. J. Org. Chem. 62, 5458-5470 (1997). Compounds 8 to 12 were also synthesized according to this protocol (see synthesis diagram in FIG. 2B).

Buffers

All the experiments were carried out in a 10 mM sodium cacodylate buffer of pH 7.6 containing 0.1 M of lithium chloride (or sodium chloride). The absence of fluorescent contamination in the buffer was verified beforehand. The fluorescent oligonucleotide is added at the final concentration of 0.2 μM.

FRET Method

The identification of G-quartets ligands was carried out by fluorescence according to the FRET (Fluorescence Resonance Energy Transfer) method which corresponds to a dipole-dipole resonance interaction between two molecules close to each other, one of which, the donor, transfers its excitation energy to the other molecule, or acceptor. The formation of an intramolecular G-quartet structure must bring the 2 chromophores close enough to be able to observe a transfer of energy.

Fluorescence Studies

All the fluorescence measurements were carried out on a Spex Fluorolog DM1B apparatus, using an excitation line width of 1.8 nm and an emission line width of 4.5 nm. The specimens are placed in a micro quartz dish measuring 0.2×1 cm. The temperature of the specimen is controlled by an external water bath. The single oligonucleotide was analysed at 20, 30, 40, 50, 60, 70 and 80° C. The emission spectra are recorded using an excitation wavelength of 470 nm. The excitation spectra are recorded using either 515 nm, or 588 nm as emission wavelength. The spectra are adjusted to take account of the response of the instrument by reference curves. A substantial extinction (80-90%) of the fluorescence of the fluorescein at ambient temperature is observed, in keeping with an intramolecular folding of the oligonucleotide at 20° C. in the form of a G-quadruplex, which induces a juxtaposition of its 5′ and 3′ ends, respectively bound to the fluorescein and to the tetramethylrhodamine (tamra in short). This juxtaposition involves a phenomenon already described, extinction of fluorescence of the donor by FRET, and a sensitized emission of the acceptor.

Tm in Fluorescence

A stock solution of oligonucleotide at the strand concentration of 0.2 μM in a 0.1 M LiCl 10 mM cacodylate buffer of pH 7.6 is prepared beforehand, heated briefly to 90° C. and cooled slowly to 20° C., then distributed by aliquots of 600 μl in the fluorescence dishes. 3 μl of water (for the control) or 3 μl of the product to be tested (stock at 200 μM, final concentration 1 μm) are then added and mixed. The specimens are then left to incubate for at least 1 hour at 20° C. before each measurement. The use of longer incubation times (up to 24 hours) does not affect the result obtained.

Each experiment allows only a single specimen to be measured. This is firstly incubated at an initial temperature of 20° C., raised to 80° C. in 40 minutes, left for 5 minutes at 80° C., then cooled to 20° C. in 62 minutes. During this time, the fluorescence is measured simultaneously at two emission wavelengths (515 nm and 588 nm) using 470 nm as excitation wavelength. A measurement is carried out every 30 seconds. The temperature of the water bath is recorded in parallel, and the fluorescence profile as a function of the temperature is reconstituted from these values. The fluorescence profiles are then standardized between 20° C. and 80° C., and the temperature for which the emission intensity at 515 nm is the average of those and high and low temperature is called Tm. In these conditions, the Tm of the reference specimen without addition of product is 44° C. in a lithium chloride buffer. This temperature is increased to more than 55° C. in the sodium chloride buffer. The addition of a compound stabilizing the G-quadruplex induces an increase in the Tm. This increase is considered significant if it is greater than 3° C.

The anti-telomerase biological activity is determined by the following experimental protocol:

Preparation of the Extract Enriched in Human Telomerase Activity

The HL60 leukemia line was obtained from the ATCC (American Type Culture Collection, Rockville USA). The cells are cultured in suspension in RPMI 1640 medium containing 2 mM L-glutamine, 200 U/ml penicillin, 200 μg/ml streptomycin, 50 μg/ml gentamycin with an added 10% heat-inactivated foetal calf serum.

An aliquot of 10⁵ cells is centrifuged at 3,000×G and the supernatant liquid removed. The cell pellet is re-suspended by several successive pipettings in 200 μl of lysis buffer containing 0.5% CHAPS, 10 mM Tris-HCl of pH 7.5, 1 mM MgCL₂, 1 mM EGTA, 5 mM 62-mercaptoethanot, 0.1 mM PMSF and 10% glycerol and is preserved in ice for 30 minutes. The lysate is centrifuged at 16,000×G for 20 minutes at 4° C. and 160 μl of supernatant liquid is recovered. The determination of the proteins of the extract is effected by the Bradford method, the extract is preserved at −80° C.

Determination of the Telomerase Activity

The inhibition of the telomerase activity is determined by an extension protocol for the TS oligonucleotide (^{5′}AATCGTTCGAGCAGAGTT^{3′}), in the presence of a cell extract enriched in telomerase activity and compounds which are added at different concentrations (10, 1, 0.1 and 0.1 μg/ml). The extension reaction is followed by a PCR amplification of the extension products with the help of the TS and CXext nucleotides (^{5′}GTGCCCTTACCCTTACCCTTACCCTAA^{3′}).

The reaction medium is prepared according to the following composition:

| Tris HCl pH 8.3 | 20 | mM |
|---|---|---|
| MgCl₂ | 1.5 | mM |
| Tween 20 | 0.005% | (W/V) |
| EGTA | 1 | mM |
| dATP | 50 | μM |
| dGTP | 50 | μM |
| dCTP | 50 | μM |
| dTTP | 50 | μM |
| TS oligonucleotide | 2 | μg/ml |
| CXext oligonucleotide | 2 | μg/ml |
| Bovine serum albumin | 0.1 | mg/ml |
| Taq DNA polymerase | 1 | U/ml |
| alpha ³²P dCTP (3,000 Ci/mmol) | 0.5 | μl |
| Telomerase extract | 200 ng under a | |
| | volume of 10 μl | |
| Product to be tested or solvent | under a volume of 5 μl | |
| Double distilled water QD | 50 | μl |

The oligonucleotides are obtained from Eurogentec (Belgium) and are preserved at −20° C. at a stock concentration of 1 mg/ml in distilled water.

The reaction specimens are collected in 0.2 ml PCR tubes and a drop of paraffin oil is placed on each of the reactions of the experiment before the tubes are sealed.

The reaction specimens are then incubated in a Cetus 4800 type apparatus according to the following temperature conditions:

15 minutes at 30° C.,

1 minute at 90° C.,

followed by 30 cycles of

30 seconds at 94° C.,

30 seconds at 50°,

and 1 minute 30 seconds at 72° C.,

followed by a final cycle of 2 minutes at 72° C.

For each of the specimens, an aliquot of 10 μl is pipetted under the layer of oil and mixed with 5 μl of a deposit buffer containing:

| TBE | 3X |
|---|---|
| Glycerol | 32% (W/V) |
| Bromophenol blue | 0.03% |
| Xylene cyanole | 0.03% |

The specimens are then analysed by electrophoresis in 12% acrylamide gel in a 1× TBE buffer for 1 hour under a current of 200 volts, using a Novex electrophoresis system.

The acrylamide gels are then dried on a sheet of 3 MM Whatmann paper at 80° C. for 1 hour, analysed and quantified.

For each concentration of compound tested, the results are expressed as a percentage inhibition of the reaction and calculated from the non-treated enzymatic control and the specimen without enzyme (blank) according to the following formula:

(Compound value−blank value/Enzymatic control value−blank value)×100.

The concentration of compound inducing a 50% inhibition of the telomerase reaction (IC₅₀) is determined using a semi-logarithmic graphic representation of the inhibition values obtained as a function of each of the tested compound.

A compound is considered active as an anti-telomerase agent when the quantity inhibiting 50% of the telomerase reaction is noticeably below 5 μM.

Specific G-4 Ligands

In the tests for which the results are reported below, two oligonucleotides were used of sequences SEQ ID No. 1 and SEQ ID No. 2, replaced at the 5′ end by a fluorescein (fluo in short) group and at 3′ end by a tamra group, conforming to the following sequences:

fluo-GGGTTAGGGTTAGGGTTAGGG-tamra,
fluo-TTGGGTTAGGGTTAGGGTTAGGG-tamra

13 compounds of dibenzophenanthrolines were tested. Their formulae are given FIG. 2A.

Compounds 1 to 7 were compared at a colorant concentration of 1 μM. The results summarized in Table 1.

| | | | |
|---|---|---|---|
| | Δ Tm G4(FRET)(° C.) | IC₅₀ Telomerase | Δ Tm triplex^{a} |
| Compounds | 1 μM colorant | (μM) | 15 μM colorant |

| | | | |
|---|---|---|---|
| 1 | +11.5 | 0.3 | 20 |
| 2 | +5.5 | 1.45 | 37 |
| 3 | +9.5 | 0.75 | 44 |
| 4 | +11.5 | 1.0 | 18 |
| 5 | +12.5 | 0.5 | 7 |
| 6 | +10 | 0.9 | 16 |
| 7 | +2.5 | 2.0 | n.a. |
| 8 | +15 | 0.46 | |
| 9 | +10.5 | 0.95 | |
| 10 | +8.4 | n.a. | |
| 11 | +18 | 0.13 | |
| 12 | +7 | 0.48 | |
| 13 | +19.7 | 0.028 | |

^{a}values deduced from the observations reported by Baudoin et al in Chemistry cited above

These results confirm the binding of the compounds of dibenzophenanthrolines to the quadruplex DNA.

In identical conditions, tetra [N-methyl pyridyl]porphyrin and a 2,6-disubstituted dianthraquinone give stabilizations of approximately 4° C.

These values of Δ Tm are significantly smaller than those obtained with most of the ligands of dibenzophenanthrolines. It will be noted in particular that the Δ Tm of ligand 5 is +12.5° C. and that of ligand 13 is +19.7° C.

The effect of Δ Tm was compared with the telomerase inhibition efficiency.

A TRAP (Telomerase Repeat Amplification Protocol) test was carried out on compounds 1 to 7 in identical conditions, operating according to Krupp and et al Nucleic Acids Res. 25, 919-921 (1997). A lysate of HL60 cells was used as source of telomerase. The TRAP reaction mixture was added directly to the mixture of compound and telomerase extract.

An amplification by PCR as described above was then carried out.

The telomerase elongation products were migrated onto a 12% polyacrylamide gel in non-denaturing conditions.

FIG. 3A relates to the in vitro inhibition of the telomerase by compounds 1 and 2, where B corresponds to tests without nuclear extracts and E, in the presence of telomerase-active nuclear extracts. Compounds 1 and 2 were tested at 3 different concentrations: 0.1, 1 and 10 μM, from right to left.

FIG. 3B gives the correlation between the in vitro inhibition of the telomerase (axis Y, expressed as concentration necessary to obtain a 50% inhibition of the telomerase activity in a standard TRAP test) and the G4 stabilization (axis Y, expressed in Δ Tm of the oligonucleotide with SEQ ID No. 1). The results correspond to an average of at least 2 independent experiments.

Examination of FIG. 3A shows, quite particularly as far as compound 1 is concerned, that increasing concentrations lead to the disappearance of the bands with less mobility, corresponding to the telomerase elongation products. It will be seen that compound 2 also inhibits the telomerase. The results given in table 1 are illustrated by FIG. 3B which shows the relationship between telomerase inhibition efficiency and Δ Tm. It will be seen that the compounds which effectively inhibit telomerase in vitro, quite particularly compounds 1, 3, 4, 5, 6 and 13, all stabilize the G-quartet structure by more than 9° C. Compounds 2 and 7 have IC₅₀ values of 1.4 and 2 μM respectively.

Antiproliferative Effect of Compounds of Dibenzophenanthrolines

Cells of cancer lines are incubated for 3 days in the presence of different concentrations of compound 1.

The cytotoxicity of compound 1, the most active in relation to telomerase, was tested on the human HeLa tumoral line.

These cells are cultured in a MEM (Life Technologies) medium containing 10% of decomplemented foetal calf serum, glutamine, nonessential amino acids (1%) and antibiotics (penicillin and streptomycin). The ligand is diluted in the culture medium after adhesion of the cells on 96-well plates (2500 cells per well). The number of cells is estimated by the “Cell Titer 96 Aqueous One Solution Cell Proliferation Assay” kit from Promega. Each measurement was carried out in quadruplicate. The cells are left in the presence of the compound for 1 to 4 days, then counted.

The IC₅₀ measured for compound 1 is 0.5 micromolar on the HeLa cells placed in the presence of compound 1 for 24 hours. A 100% mortality rate is observed for concentrations of 5 micromolars or more. The toxicity of product 1 is reinforced when the cells are incubated for 96 hours in its presence (100% mortality at 0.5 micromolars).

1. Use of aromatic compounds capable of binding to G-quadruplex structures in order to prepare medicaments having an anti-telomerase effect, characterized in that the said compounds conform to Formula (I) 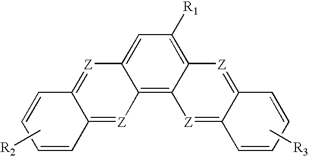
in which
R₁, R₂ and R₃, identical to or different from one another, represent a hydrogen atom, or a —CH₂—NH—(CH₂)ₙ—X group, in which n is an integer from 2 to 4, and X is chosen from among the —NH₂, —N(CH₃)₂ radicals, a heterocyclic radical such as the piperidyl, imidazolyl, morpholinyl radical, or a condensed heterocyclic radical of indole type,
Z represents CH or N, each compound containing two nitrogen atoms in the “Z” positions.

2. Use according to claim 1 of a derivative of Formula (II)

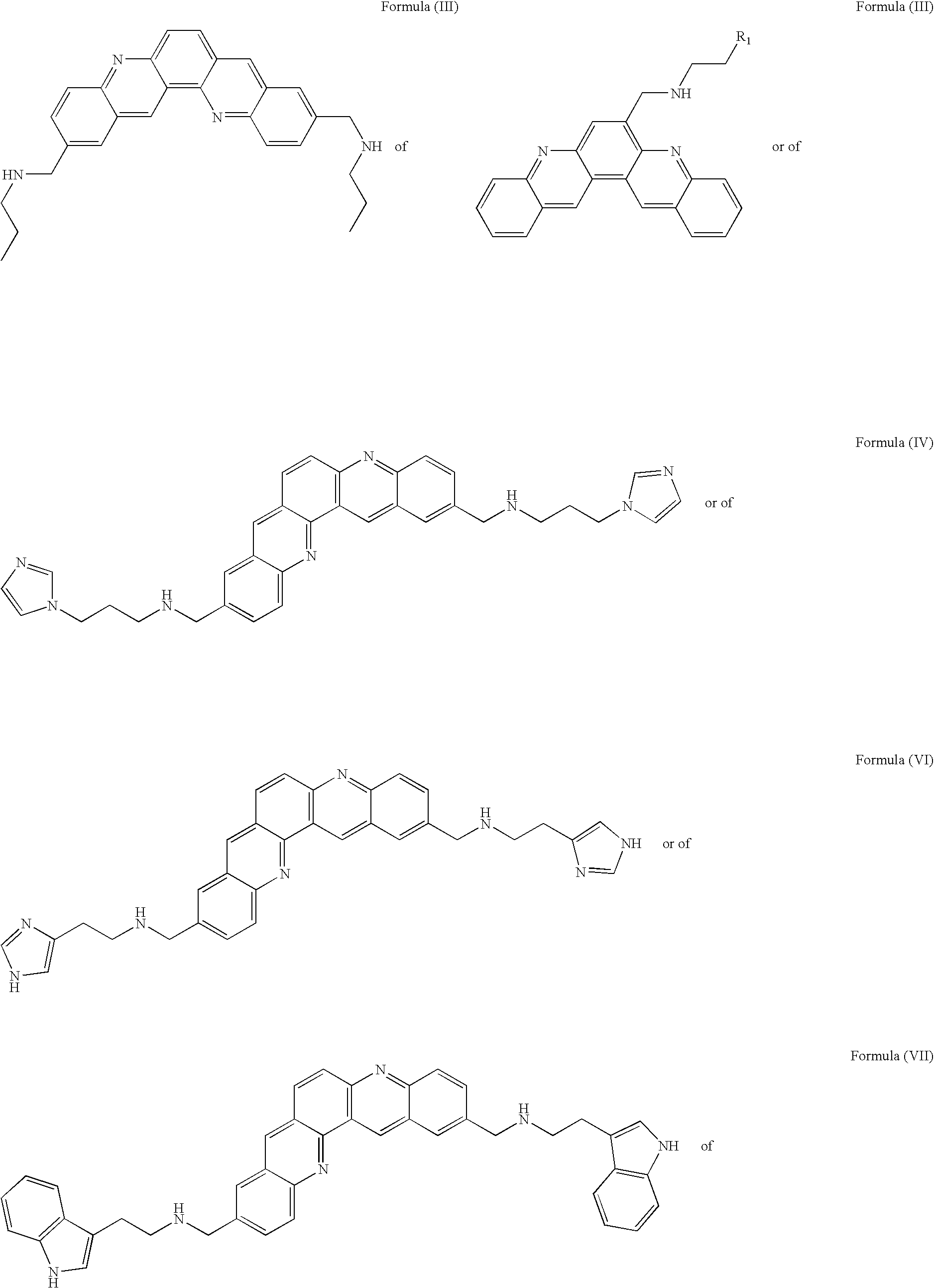

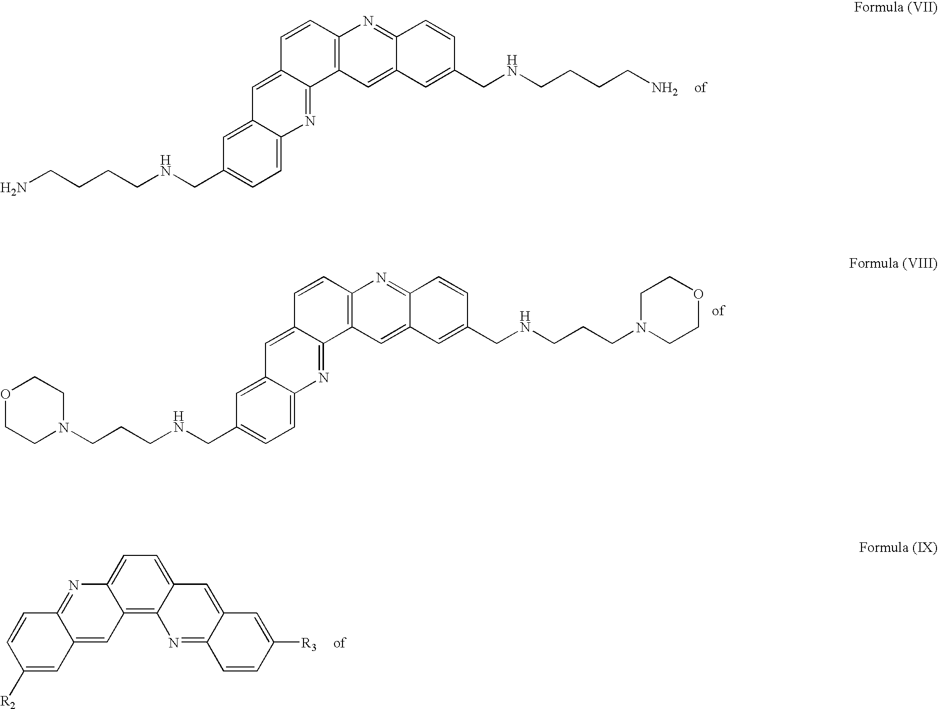

3. Novel dibenzophenanthrolines, characterized in that they conform to Formula (IV) 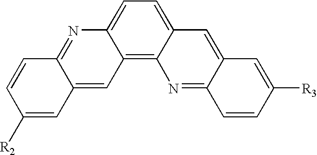
in which R₁ and R₃ are identical and represent a —CH₂—NH—(CH₂)ₙ—X group, in which n is an integer from 2 to 4, and X is chosen from among the —NH₂, —N(CH₃)₂ radicals, a heterocyclic [radical] such as the piperidyl, imidazolyl radical, or a condensed heterocyclic radical of indole type.

4. Pharmaceutical compositions, characterized in that they contain a therapeutically effective quantity of at least one compound according to claim 3, in combination with a pharmaceutically inert vehicle.

5. Use of compounds of Formula I according to claim 1, to prepare medicaments for an administration by the oral, nasal, buccal, injectable, parenteral, rectal, vaginal or topical route.
